(19) United States
(12) Patent Application Publication
(10) Pub. No.: US 2015/0094550 A1
(43) Pub. Date: Apr. 2, 2015

(54) DUAL-SPECTRA PULSE OXIMETER SENSORS AND METHODS OF MAKING THE SAME

(71) Applicant: General Electric Company, Schenectady, NY (US)

(72) Inventors: Jason Harris Karp, Niskayuna, NY (US); Ming Yin, Rexford, NY (US)

(73) Assignee: General Electric Company, Schenectady, NY (US)

(21) Appl. No.: 14/040,789

(22) Filed: Sep. 30, 2013

(51) Int. Cl. A61B 5/024; A61B 5/1455

(52) U.S. Cl. CPC A61B 5/02433; A61B 5/14552
USPC 600/324; 29/832

(57) ABSTRACT

A dual-spectra pulse oximeter sensor is provided. The dual-spectra pulse oximeter sensor includes a sensor substrate, an array of a plurality of pairs of wavelength sources disposed on the sensor substrate, and a plurality of wavelength conversion devices. Further, each pair of the plurality of pairs of wavelength sources comprises a pair of light emitting diodes. Moreover, one light emitting diode of each pair of light emitting diodes is operatively coupled to a wavelength conversion device of the plurality of wavelength conversion devices.

### BACKGROUND

The invention relates to dual-spectra sensors, and more particularly to dual-spectra sensors for use in pulse oximeters.

Early detection of low blood oxygen is critical in a wide variety of medical applications. For example, when a patient receives an insufficient supply of oxygen in critical care and surgical applications, brain damage and death can result in a matter of minutes. Oximetry is used to study and measure the oxygen status of blood in the body. Pulse oximetry is a non-invasive procedure for measuring the oxygen saturation level of arterial blood, an indicator of the oxygen status of the blood. A pulse oximeter relies on a sensor attached to a patient in order to measure the blood oxygen saturation. In practice, light is passed through a portion of a patient's body which contains arterial blood flow. An optical sensor is used to detect the light which has passed through the body, and variations in the detected light at various wavelengths are then used to determine arterial oxygen saturation and/or pulse rates. Oxygen saturation may be calculated using an absorption equation based on Beer's Law.

Typically, pulse oximeters use dual light sources having different emission spectra to measure differential absorption spectra of blood. The absorption difference during systolic and diastolic cardiac states may be used to determine the blood oxygenation concentration and is a critical health monitoring parameter. Generally, pulse oximeters use red and infrared light emitting diodes to probe oxygenated and deoxygenated hemoglobin absorption.

In operation, the pulse oximeters are typically attached to a patient's body part having a blood flow. By way of example, the pulse oximeters may be attached to a finger, earlobe, or foot of the patient. In instanced where the pulse oximeter is attached to a finger, a sensor of the pulse oximeter is disposed on one side of the finger such that emitters of the sensor project light through outer tissues of the finger and into blood vessels and capillaries present in the finger. A photodiode is positioned opposite to the emitters and is configured to detect emitted light emerging from the outer tissues of the finger. The photodiode generates a signal based on the detected emitted light. The photodiode then relays that signal to a processor of the pulse oximeter. The processor determines blood oxygen saturation by computing a differential absorption by the arterial blood of the two wavelengths (e.g., red and infrared) emitted by the sensor.

With the increase in demand for home healthcare solutions, it is desirable that cost of monitoring devices, such as a pulse oximeter, be reduced to make such monitoring devices more affordable. Primarily, one of the reasons behind the relatively high cost of such monitoring devices is the fabrication and assembling methods used to make these devices. For example, in case of a pulse oximeter, typically, the light emitting diodes of different lengths are assembled on a substrate using heterogeneous assembling methods. The heterogeneous assembling methods, such as magnetically directed self-assembly, are time consuming as compared to their homogeneous counterparts. Further, probability of the heterogeneous assembling methods having a defect in the assembly process are much higher relative to the homogeneous methods, where similar devices need to be positioned at determined locations.

### BRIEF DESCRIPTION

In one embodiment, a dual-spectra pulse oximeter sensor is provided. The dual-spectra pulse oximeter sensor includes a sensor substrate, an array of a plurality of pairs of wavelength sources disposed on the sensor substrate, and a plurality of wavelength conversion devices. Further, each pair of the plurality of pairs of wavelength sources comprises a pair of light emitting diodes. Moreover, one light emitting diode of each pair of light emitting diodes is operatively coupled to a wavelength conversion device of the plurality of wavelength conversion devices.

In another embodiment, a pulse oximetry system is provided. The system includes a pulse oximeter device having a dual-spectra pulse oximeter sensor and an optical detector for detecting emission signals from the dual-spectra pulse oximeter sensor. The dual-spectra pulse oximeter sensor includes a sensor substrate, an array of a plurality of pairs of wavelength sources disposed on the sensor substrate, and a plurality of wavelength conversion devices. Further, each pair of the plurality of pairs of wavelength sources comprises a pair of homogeneous light emitting diodes. Moreover, one diode of each pair of homogeneous light emitting diodes is operatively coupled to a wavelength conversion device of the plurality of wavelength conversion devices. The system further includes a processor for processing the detected emission signals and a display member configured to display processed data from the processor.

In yet another embodiment, a method of making a dual-spectra pulse oximeter sensor is provided. The method includes providing a sensor substrate, providing a plurality of light emitting diodes, and positionally disposing each of the plurality of light emitting diodes on the substrate to form a plurality of pairs of the light emitting diodes. The method further includes selectively operatively coupling one light emitting diode corresponding to each pair of the plurality of pairs of light emitting diodes to a corresponding wavelength conversion device of a plurality of wavelength conversion devices.

### DRAWINGS

These and other features and aspects of embodiments of the invention will become better understood when the following detailed description is read with reference to the accompanying drawings in which like characters represent like parts throughout the drawings, wherein:

FIG. 1 is a cross-sectional view of a portion of a dual-spectra device employing a pair of wavelength sources, where the pair of wavelength sources includes a pair of homogeneous light emitting diodes with one light emitting diode of the pair of homogeneous light emitting diodes being operatively coupled to a wavelength conversion device, in accordance with aspects of the present disclosure;

FIG. 2 is a schematic representation of a dual-spectra pulse oximeter sensor having an array of a plurality of wavelength sources, in accordance with aspects of the present disclosure;

FIG. 3 is a block diagram of an example pulse oximetry system employing the pulse oximeter sensor of FIG. 1, in accordance with aspects of the present disclosure; and

FIG. 4 is a flow diagram of an example method of making the dual-spectra pulse oximeter sensor of FIG. 1, in accordance with aspects of the present disclosure.

### DETAILED DESCRIPTION

Embodiments of the present disclosure relate to dual-spectra pulse oximeter sensors and methods of making the same. Dual-spectra generation using photoluminescent devices, such as energy conversion devices, enables a single type emitter device to produce spectral peaks in different spectral ranges. In certain embodiments, a dual-spectra pulse oximeter sensor may include a sensor substrate and an array of a plurality of pairs of wavelength sources. In some of these embodiments, the wavelength sources may include a pair of light emitting diodes. In one embodiment, the pair of light emitting diodes may include homogeneous light emitting diodes. In certain embodiments, the term “homogeneous light emitting diodes” may be used to refer to diodes that may not have any detectable difference in structure, emitted wavelength range, and other optical and physical properties. Moreover, in one example, the homogeneous light emitting diodes may be single-wavelength diodes. In some embodiments, one of the wavelength sources may be adapted to emit a wavelength range that is different from an original wavelength range emitted by the corresponding light emitting diode. Accordingly, the pair of wavelength sources having the homogeneous light emitting diodes may be configured to emit two different wavelength ranges. In one example, the homogeneous light emitting diodes of the dual-spectra pulse oximeter sensor may employ a pair of light emitting diodes configured to emit light in the red wavelength range. In this example, one of the red light emitting diodes may be modified to emit light in the infrared wavelength range.

In certain embodiments, the dual-spectra pulse oximeter sensor may include a plurality of wavelength conversion devices. In one example, the wavelength sources may include homogeneous light emitting diodes, and one of the light emitting diodes of a pair of homogeneous light emitting diodes may be operatively coupled to a wavelength conversion device. In these embodiments, the pair of homogeneous light emitting diodes is configured to emit light at two different wavelengths or wavelength ranges. In one embodiment, one of the diodes of the pair of diodes may emit through a wavelength conversion device that emits at a lower wavelength range. The light emitting diode coupled to a wavelength conversion layer may emit through the wavelength conversion layer of the wavelength conversion device. It should be noted, dual emission wavelengths may be generated from a pair of homogeneous light emitting diodes in instances where the converted wavelength is of a lower energy. In one embodiment, one of the light emitting diodes of a pair of homogenous light emitting diodes configured to emit in the red wavelength range may be operatively coupled to the wavelength conversion layer to form a pair of wavelength sources, where one wavelength source is configured to emit in the red wavelength range and another wavelength source is configured to emit in the infrared wavelength range.

Non-limiting examples of the wavelength conversion device may include a phosphor based material. In a particular example, the wavelength conversion device may include a phosphor layer. In some embodiments, the wavelength sources may include red and infrared sources, whereas in some other embodiments, the wavelength sources may include blue and green wavelength sources. Ultraviolet, blue or green emitters may be used to in conjunction with phosphors to generate all the requisite wavelengths needed for pulse oximetry or other dual-spectra applications.

In certain embodiments, the dual-spectra pulse oximeter sensor is configured for use in pulse oximetery. In one embodiment, the dual-spectra pulse oximeter sensor of the present disclosure is configured for use with multiple oximeter systems. Moreover, in some embodiments, the dual-spectra pulse oximeter sensor may be retrofitted in existing pulse oximetry systems with minimal or no adjustments required for the system or the pulse oximeter sensor.

Additionally, in one embodiment, only one wavelength source of each pair of the plurality of wavelength sources may employ a wavelength conversion device. In another embodiment, both the wavelength sources of a pair of wavelength sources may employ wavelength conversion devices. Moreover, the wavelength sources may include two or more wavelength conversion devices. In certain embodiments, the wavelength sources may include diodes, such as, but not limited to, light emitting diodes. Non-limiting examples of the light emitting diodes may include organic light emitting diodes, inorganic light emitting diodes, quantum dot light emitting diodes, or combinations thereof.

In certain embodiments, the pulse oximeter sensor may employ red and infrared wavelength sources. It should be noted that the absorption contrast between oxygenated and deoxygenated hemoglobin is relatively high at red wavelengths but is much lower at infrared wavelengths. In a two wavelength pulse oximeter sensor, a narrow bandwidth 660 nm source configured to emit in red wavelength range may be converted to an infrared source using an infrared phosphor. Phosphor emission is typically broader in spectrum and is most suitable where the hemoglobin shows minimal absorption contrast. In a particular example, the pair of wavelength sources may be formed using a pair of homogeneous light emitting diodes configured to emit in the red wavelength range, and a wavelength conversion layer may be disposed on one of the light emitting diodes to decrease the energy of the emitted wavelengths to the infrared wavelength range. In this example, the pair of wavelength sources may emit light in the red and infrared wavelength ranges.

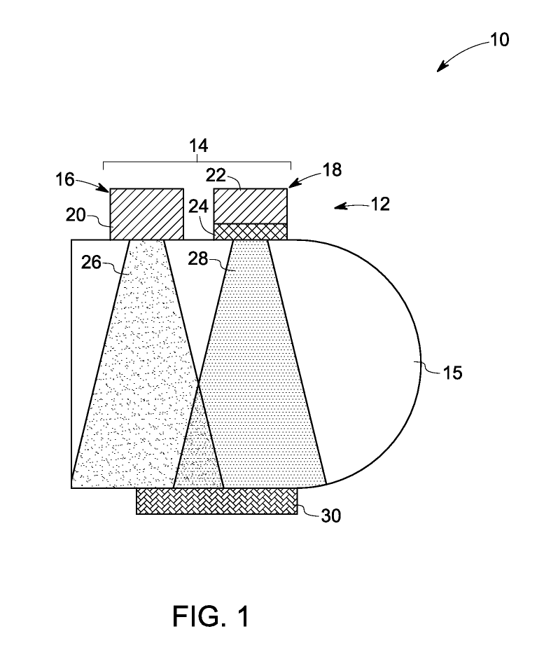

FIG. 1 illustrates a portion 10 of an example dual-spectra pulse oximeter sensor 12. In the illustrated embodiment, the dual-spectra pulse oximeter sensor 12 is disposed on a fingertip 15 of a patient (not shown). The dual-spectra pulse oximeter sensor 12 may include a pair 14 of wavelength sources 16 and 18. The wavelength source 16 may include a light emitting diode 20 and the wavelength source 18 may include a light emitting diode 22. The light emitting diodes 20 and 22 may be homogeneous diodes that are configured to emit light in the same or substantially similar spectral regions or wavelength regions. In one embodiment, the light emitting diodes 20 and 22 may be configured to emit light in a first spectral region. In one example, the light emitting diodes 20 and 22 may be configured to emit in the red wavelength region. The light emitted by the wavelength source 16 is generally represented by reference numeral 26.

In certain embodiments, the second wavelength source 18 may include the light emitting diode 22 and a wavelength conversion device 24. The wavelength conversion device 24 may be configured to modulate the spectral characteristics of the light emitting diode 22. Resultant emitted light 28 from the wavelength source 18 may have spectral characteristics that are different from the spectral characteristics of the light 26 emitted by the wavelength source 16. A portion of the emitted lights 26 and 28 may be received by an optical detector 30. The optical detector 30 may be configured to detect at least a portion of light emitted by the two wavelength sources 16 and 18. The optical detector 30 may be integral or external to the sensor 12.

Furthermore, in one example, the wavelength conversion device 24 may include a layer of a wavelength conversion material. The wavelength conversion material may be such that when the light emitted by the diode 22 excites the wavelength conversion material, the wavelength conversion material emits a light that has a wavelength range different from the wavelength range of the light emitted by the corresponding diode, while the light emitted by the diode 20 that does not include a wavelength conversion device is unaffected by the presence of the wavelength conversion device 24 in the pair 14 of the wavelength sources.

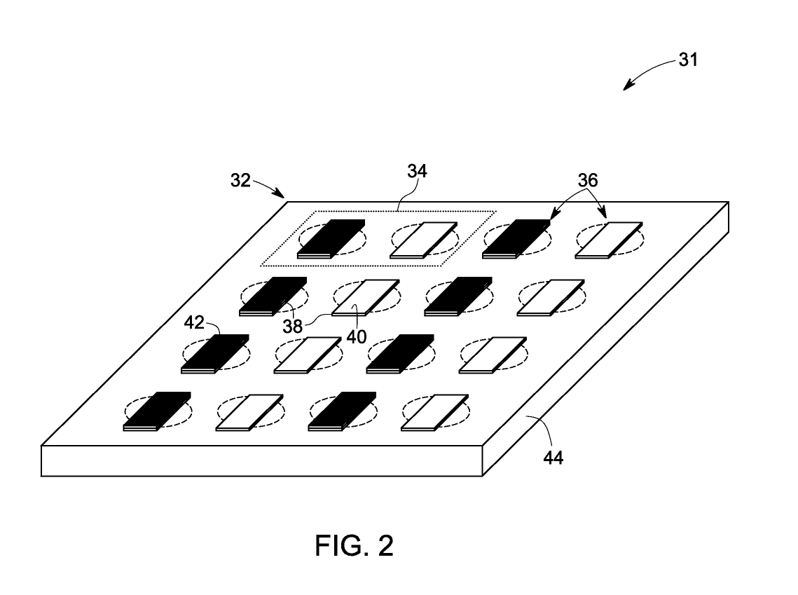

FIG. 2 illustrates an example dual-spectra pulse oximeter sensor 31 having an array 32 of a plurality of pairs 34 of wavelength sources 36. Each pair 34 having the plurality of wavelength sources 36 may in turn include a pair 38 of homogeneous diodes 40. One wavelength source 36 of each of the pairs 34 of the wavelength sources 36 may be operatively coupled to a corresponding wavelength conversion device 42. The array 32 may be disposed on a substrate 44, such as, but not limited to, a plastic substrate. In some embodiments, the substrate 44 may be transparent to the wavelengths emitted by the wavelength sources 36.

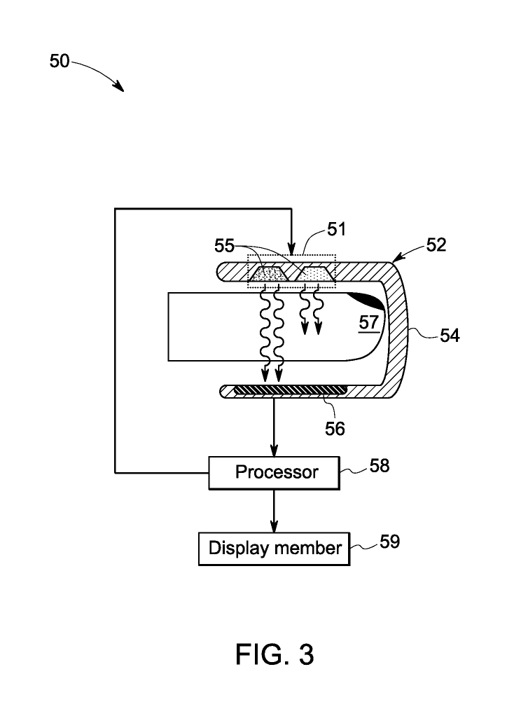

FIG. 3 illustrates an example embodiment of a system 50 employing a pulse oximeter device 52 having a dual-spectra pulse oximeter sensor 51 and an optical detector 56. The pulse oximeter device 52 includes a sensing head 54 that is configured to be coupled (e.g., clipped) to a patient's fingertip 57. The dual-spectra pulse oximeter sensor 51 may include a plurality of pairs of wavelength sources 55. Although not illustrated, in some embodiments, the pulse oximeter device 52 may be attached to the head of the patient, for example the patient's ear lobe, or forehead. Use of the head for pulse oximetry measurements is desirable in cases where blood flow is relatively low as the involuntary motion of the head is typically less than that of the hand, hence, signal to noise ratio is relatively larger for the head as compared to the hands.

The wavelength sources 55 may include homogeneous light emitting diodes (not shown). In one embodiment, the wavelength sources 55 may include a first light emitting diode configured to emit light in the red wavelength range, and a second light emitting diode configured to emit light in the infrared wavelength range. One of the light emitting diode of the wavelength sources 55 may be operatively coupled to a wavelength conversion device (not shown) to tailor a wavelength range of the light emitted from that light emitting diode.

In the illustrated embodiment, the optical detector 56 is mounted in the sensing head 54 of the pulse oximeter device 52 along with the wavelength sources 55 to detect the light or optical energy transmitted through, or reflected from the patient's tissue. In one embodiment, the optical detector 56 may be configured to determine absorption of the two wavelength ranges by synchronizing the light emitting diodes of the wavelength sources 55 in time. In one example, for relatively higher amount of hemoglobin in the blood, more light is absorbed by the arterial blood, hence, the light received by the detector 56 may be reduced. The absorption spectra of hemoglobin is relatively uniform from about 850 nm to about 940 nm making broad emission peaks acceptable. The optical detector 56 may be internal or external to the pulse oximeter sensor 52.

The absorption detected by the optical detector 56 may be processed by a processor 58. In one embodiment, the processor 58 may be configured to process the detected signals to determine in vivo blood oxygenation concentration levels using the light detected by the optical sensor 56 as an input. In one example, the processor 58 may be configured to determine the level of oxygen (SpO₂) in the blood. The processor 58 may be external or internal to the sensing head 54. The system 50 may further include a display member 59 configured to display a blood saturation level. In one embodiment, the display may be a light emitting diode display. The display member 59 may be integral part of the pulse oximeter. Alternatively, the display member 59 may be external to the pulse oximeter.

In addition to the display member 59, the system 50 may also include a provision for alarm management. For example, if the blood saturation level is below a determined limit, the system 50 may be configured to communicate an alarm to the care provider, without distressing the patient. For example, in case of a patient on oxygen support, a management system may be configured to alert the care provider with a specific display indicative of lower than desirable blood saturation level. The care provider may then accordingly adjust the setting of the oxygen concentrator, without distressing the patient.

In certain embodiments, a method of making a dual-spectra pulse oximeter sensor may include self-assembly methods. In one embodiment, a plurality of magnets may be embedded in a template substrate to form a template for positionally disposing a plurality of pairs of homogeneous light emitting diodes in the dual-spectra pulse oximeter sensor. The plurality of magnets embedded in the template substrate may be magnetized. In one example, the plurality of magnets may be magnetized using pulse magnetization methods. During fabrication of the pulse oximeter sensor, when the light emitting diodes are randomly disposed on the sensor substrate disposed on the template, self-assembly occurs when randomly disposed diodes are re-distributed by applying magnetic attractive forces.

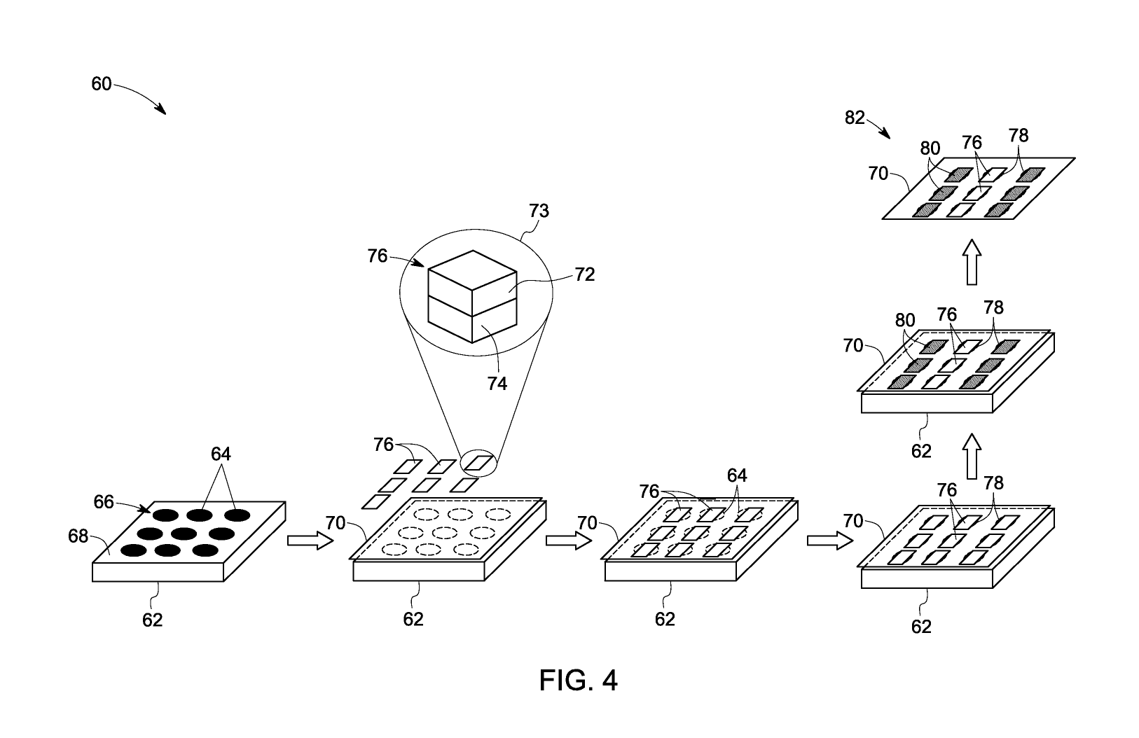

FIG. 4 illustrates a flow diagram 60 for an example method of making the dual-spectra pulse oximeter sensor such as the dual-spectra pulse oximeter sensor of FIG. 2 of the present disclosure. The method includes providing a magnetic template 62 having spatially arranged independent magnets 64 in a magnetic array 66 on a surface 68 of the template 62. In one embodiment, diced or printed magnets may be used to form the magnetic array 66 on the magnetic template 62. Non-limiting examples of the magnets 64 may include NdFeB, Sm₂Co₁₇, Fe₃O₄, or combinations thereof. In one example, the magnets 64 may be hard magnets.

Further, the method may further include disposing a sensor substrate 70 on the magnetic template 62. Additionally, the method includes providing a plurality of homogeneous light emitting diode structures 76. As illustrated in an enlarged view generally represented by reference numeral 73, each of the diode structures 76 may include a light emitting diode 72 disposed on the magnetic material 74. The diode structures 76 having a coating of the magnetic material 74 may be disposed on the sensor substrate 70, where the sensor substrate 70 is disposed on a magnetic template 62. Non-limiting examples of the sensor substrate 70 may include a flex layer and patterned interconnects on flex. In one embodiment, the sensor substrate 70 may include polymeric material such as, but not limited to, polyimide, polyethylene terapthalate, styrene polymer, polypropylene, thermoplastic polyurethane, or combinations thereof.

Subsequent to randomly disposing the diode structures 76 on the sensor substrate 70, the diode structures 76 may be positionally disposed on independent magnets 64 using magnetic attraction forces to directionally move the diode structures 76 to form a plurality of pairs of the diode structures 76 having the diodes 72. In one example, the diode structures 76 may be simultaneously disposed in their respective positions on the sensor substrate 70 in a single step. In some embodiments, exposing the diode structures 76 to the magnetic field may include bringing devices configured to produce magnetic fields in close proximity to the magnets 64. In addition to the magnetic field, the diode structures 76 may also be exposed to one or more of mechanical vibrations, acoustic and/or ultrasound vibrations, radiation, and gravitational forces to facilitate positioning of the diode structures 76 on the sensor substrate 70.

Next, the diode structures 76 may be coupled to the sensor substrate 70 at respective positions on the sensor substrate 70. Physically coupling each of the diode structures 76 to a respective position on the sensor substrate 70 may include using coupling techniques, such as, but not limited to, soldering, conductive epoxy based adhesion, under-fill material coupling, or combinations thereof. In the illustrated embodiment, the diode structures 76 may be coupled to the sensor substrate 70 using an adhesive 78. Optionally, the sensor substrate 70 having the diode structures 76 may be subjected to curing. The curing may be performed at a determined temperature for a determined period of time. Subsequently, a corresponding wavelength conversion device 80 of the plurality of wavelength conversion devices may be selectively provided. For example, the wavelength conversion devices 80 may be operatively coupled to selected one or more diodes 72 of the diode structure 76. In one embodiment, the step of selectively providing the wavelength conversion device 80 to the one or more diodes 72 may include using a mask to cover other light emitting diodes. In certain embodiments, the wavelength conversion device 80 may be disposed on the diode using techniques, such as, but not limited to, printing or laminating. In one embodiment, the step of selectively disposing the wavelength conversion devices may include simultaneously disposing the plurality of wavelength conversion devices. Subsequent to selectively disposing the wavelength conversion device 80 the mask may be removed. Next, the magnetic template 62 may be removed to form the dual-spectra pulse oximeter sensor 82.

Advantageously, an array of such homogeneous diodes may be assembled in a single step. Fabricating spectrally separated wavelength emission bands from a common source type has assembly and packaging advantages that translate to cost reduction. For example, in certain embodiments, the method of making the dual-spectra pulse oximeter sensor may include parallel processing at various stages during the fabrication of the sensor. In particular, the step of positionally disposing the diode structures 76 having the homogeneous light emitting diodes may be simultaneously performed for all the diode structures 76. Further, the step of selectively disposing the wavelength conversion devices 80 may be performed as a single step for the selected diodes. Parallel processing at various stages reduces fabrication cost while increasing the volumes being fabricated.

In accordance with systems and methods of the present disclosure, spectral properties of the diodes may be tailored to a specific wavelength band by employing currently available time efficient techniques, such as, but not limited to, masking, printing, spraying, and the like. The wavelength conversion circumvents the use of wavelength sources formed from different semiconductor compounds and having different polarity/drive requirements. Additionally, using homogeneous diodes as wavelength sources advantageously enables the dual-spectra pulse oximeter sensor to be assembled in a time efficient and economical manner as unlike assembly requirements for heterogeneous devices, the assembly requirements for homogeneous devices are less demanding. Moreover, the assembly steps may be performed in parallel. For example, instead of placing the diodes on a substrate in a sequential manner, the homogeneous diodes may be disposed in their respective positions in a single step.

While only certain features of the invention have been illustrated and described herein, many modifications and changes will occur to those skilled in the art. It is, therefore, to be understood that the appended claims are intended to cover all such modifications and changes as fall within the scope of the invention.

1. A dual-spectra pulse oximeter sensor, comprising:
a sensor substrate;
an array of a plurality of pairs of wavelength sources disposed on the sensor substrate, wherein each pair of the plurality of pairs of wavelength sources comprises a pair of light emitting diodes; and
a plurality of wavelength conversion devices, wherein one light emitting diode of each pair of light emitting diodes is operatively coupled to a wavelength conversion device of the plurality of wavelength conversion devices.

2. The dual-spectra pulse oximeter sensor of claim 1, wherein each pair of light emitting diodes comprises homogeneous light emitting diodes.

3. The dual-spectra pulse oximeter sensor of claim 1, wherein the pair of light emitting diodes comprise diodes configured to emit light in a red wavelength range.

4. The dual-spectra pulse oximeter sensor of claim 1, wherein the wavelength conversion device comprises a layer of a wavelength conversion material.

5. The dual-spectra pulse oximeter sensor of claim 4, wherein the wavelength conversion material comprises a phosphor based material.

6. The dual-spectra pulse oximeter sensor of claim 1, wherein the light emitting diode of the pair of light emitting diodes operatively coupled to the wavelength conversion device is configured to emit light in an infrared wavelength range.

7. The dual-spectra pulse oximeter sensor of claim 1, wherein the sensor substrate is a flexible substrate.

8. A pulse oximetry system, comprising:
a pulse oximeter device, comprising:
a dual-spectra pulse oximeter sensor, comprising:
a sensor substrate;
an array of a plurality of pairs of wavelength sources disposed on the sensor substrate, wherein each pair of the plurality of pairs of wavelength sources comprises a pair of homogeneous light emitting diodes;
a plurality of wavelength conversion devices, wherein one light emitting diode of each pair of homogeneous light emitting diodes is operatively coupled to a wavelength conversion device of the plurality of wavelength conversion devices;
an optical detector configured to detect absorption of wavelength ranges emitted by the plurality of pairs of the wavelength sources;
a processor configured to process the detected signals; and
a display member configured to display processed signals from the processor.

9. The pulse oximetry system of claim 8, wherein each of the plurality of wavelength conversion devices comprises a layer of a wavelength conversion material.

10. The pulse oximetry system of claim 8, wherein one or more pairs of the plurality of pairs of wavelength sources comprise a red wavelength source and an infrared wavelength source.

11. A method of making a dual-spectra pulse oximeter sensor, comprising:
providing a sensor substrate;
providing a plurality of homogeneous light emitting diodes;
positionally disposing each of the plurality of light emitting diodes on the sensor substrate to form a plurality of pairs of the homogeneous light emitting diodes; and
selectively operatively coupling one light emitting diode corresponding to each pair of the plurality of pairs of light emitting diodes to a corresponding wavelength conversion device of a plurality of wavelength conversion devices.

12. The method of claim 11, wherein the plurality of light emitting diodes is simultaneously positionally disposed in a single step.

13. The method of claim 11, wherein the step of providing the plurality of homogeneous light emitting diodes comprises disposing a magnetic material on at least a portion of a surface of each homogeneous light emitting diodes of the plurality of homogeneous light emitting diodes.

14. The method of claim 13, further comprising:
providing a magnetic template having independent magnets spatially arranged in a magnetic array; and
disposing the sensor substrate on the magnetic template.

15. The method of claim 13, wherein the step of positionally disposing each of the plurality of homogeneous light emitting diodes comprises:
disposing the plurality of homogeneous light emitting diodes having the magnetic material on the sensor substrate; and
exposing the plurality of homogeneous light emitting diodes to a magnetic field to positionally dispose the plurality of homoegeneous light emitting diodes on the sensor substrate.

16. The method of claim 11, further comprising:
physically coupling each of the plurality of homogeneous light emitting diodes to a respective position on the sensor substrate.

17. The method of claim 11, wherein the step of selectively operatively coupling one light emitting diode corresponding to each pair of the plurality of pairs of light emitting diodes to the corresponding wavelength conversion comprises:
masking the other light emitting diode corresponding to each pair of the plurality of pairs of light emitting diodes;
applying the wavelength conversion device to the one light emitting diode of each pair of the plurality of pairs of light emitting diodes; and
removing the mask.

18. The method of claim 11, wherein the step of positionally disposing each of the plurality of light emitting diodes on the sensor substrate comprises simultaneously disposing each of the plurality of light emitting diodes.

19. The method of claim 11, wherein the step of selectively operatively coupling one light emitting diode corresponding to each pair of the plurality of pairs of light emitting diodes to a corresponding wavelength conversion device of a plurality of wavelength conversion devices comprises simultaneously disposing the plurality of wavelength conversion devices.

20. The method of claim 11, further comprising providing an epoxy based adhesive to physically couple the plurality of pairs of homogeneous light emitting diodes to the sensor substrate.
